(19) United States
(12) Patent Application Publication
(10) Pub. No.: US 2015/0224283 A1
(43) Pub. Date: Aug. 13, 2015

(54) Secondary Intravenous Line Kit and Method

(71) Applicant: Peter NAGY, Ridgewood, NJ (US)

(72) Inventor: Peter NAGY, Ridgewood, NJ (US)

(73) Assignee: EZ101 LLC, Ridgewood, NJ (US)

(21) Appl. No.: 14/617,673

(22) Filed: Feb. 9, 2015

Related U.S. Application Data
(60) Provisional application No. 61/937,783, filed on Feb. 10, 2014.

(51) Int. Cl. A61M 25/00

(52) U.S. Cl. CPC A61M 25/0043

(57) ABSTRACT

A method for administering a primary intravenous fluid from a first sterile fluid container and a second intravenous fluid from a second sterile fluid container, which method requires using only one catheter tube, thus reducing both the equipment and the labor required to use dual intravenous solutions.

### RELATED APPLICATIONS

This application claims priority from U.S. provisional patent filing Ser. No. 61/937783, filed 10 Feb. 2014, the contents of which are here incorporated by reference.

### GOVERNMENT INTEREST

None

### INTRODUCTION

Intravenous therapy is known in the art, as is using intravenous administration to administer a first or primary fluid. In addition to the “primary” intravenous fluid, a patient may also need a secondary intravenous fluid. An example is where the patient needs normal saline more or less continuously, and periodically needs administration of a therapeutic agent such as an antibiotic or chemotherapeutic liquid. To achieve this, the art teaches to use a primary intravenous line to feed the primary fluid (e.g., normal saline) more or less continuously, while a secondary intravenous line is used to periodically administer a therapeutic agent (e.g., an antibiotic or chemotherapy solution or suspension).

The prior art method to administer primary and secondary fluids to a patient intravenously is illustrated in FIG. 1. FIG. 1 shows a primary sterile fluid container (typically a flexible bag, as shown, but perhaps a bottle or other sterile vessel) [1] suspended from a hanger [2] and connected to a primary catheter tube [3] having a primary air lock [4] allowing air to escape the catheter tube before the air enters the patient's blood stream. The hanger [2] also suspends a secondary sterile fluid container [5] connected to a secondary catheter [6] having a secondary air lock [7].

### SUMMARY

I have found a way to entirely eliminate the need for a second catheter tube [6] and second air lock [7]. My approach thus eliminates the direct cost of the second catheter tube and air lock. By eliminating the need for a second catheter tube, my approach also reduces the amount of catheter tubes and air locks a health care facility will need to keep in inventory; this reduces the indirect cost of the working capital required to maintain that inventory. Eliminating the need for a second catheter tube also eliminates the need for the skilled labor (e.g., nursing labor) used to set up the second catheter tube. Further, eliminating the need for a second catheter tube eliminates any risk of contamination due to the second catheter tube: this reduces catheter contamination risk by half, and minimizes the amount of skilled labor needed to periodically flush and disinfect catheters.

### BRIEF DESCRIPTION OF THE DRAWINGS

FIG. 1 illustrates the prior art approach to administering a secondary intravenous solution.

FIG. 2 shows a primary sterile fluid container [1] suspended from a hanger [2].

FIG. 3: From the starting configuration exemplified in FIG. 2, one clamps shut the primary catheter tube [3] using a catheter tube clamp [9].

FIG. 4: The opening tube for the secondary sterile fluid container [5] is then opened.

FIG. 5: The opening of the primary sterile fluid container [1] is plugged with a sterile plug [8].

FIG. 6: The sterile plug [8] is removed from the primary sterile fluid container [1], the primary catheter tube [3] having a primary air lock [4] is disconnected from the secondary sterile fluid container [5] and re-connected to the primary sterile fluid container [1]. FIG. 7: A C-clamp dimensioned to completely close catheter tubing.

### DETAILED DESCRIPTION

To practice my method, one may refer to the Figures.

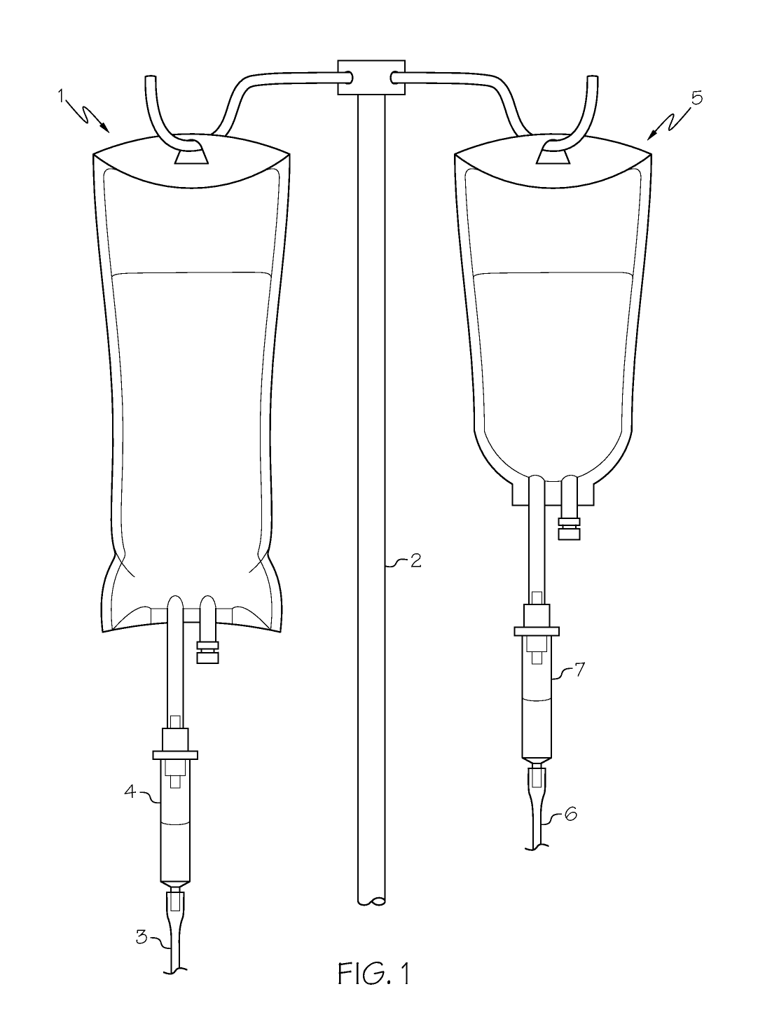

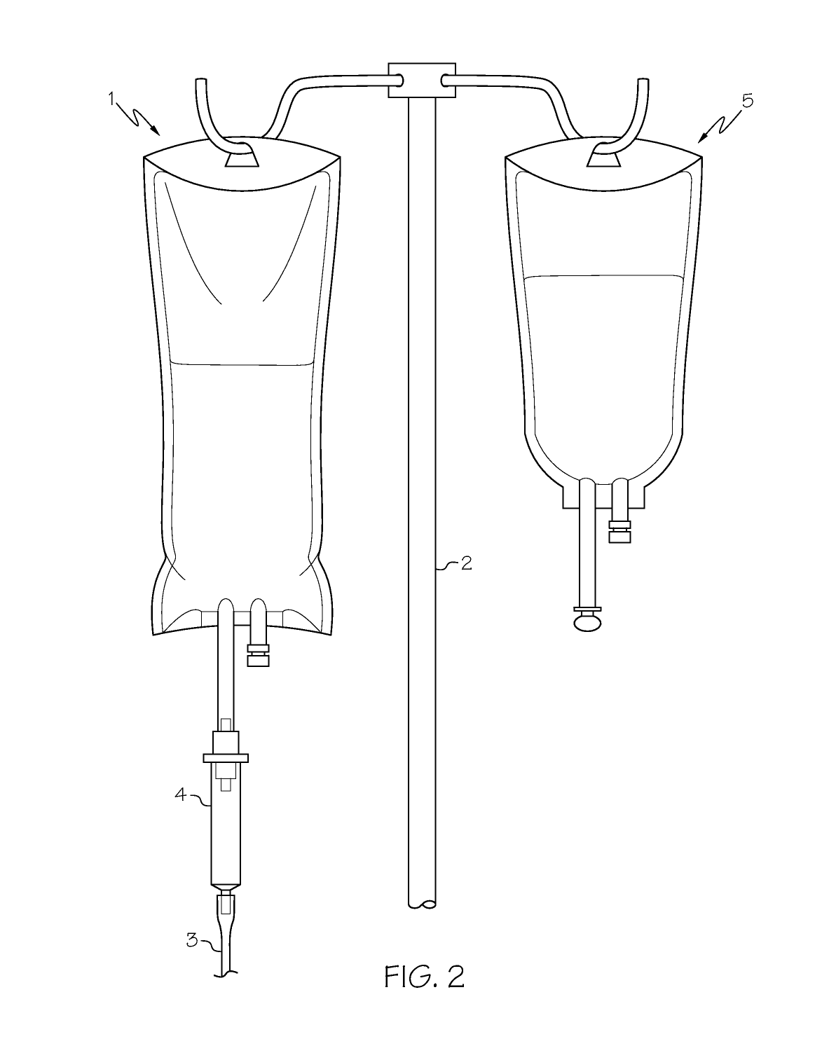

FIG. 1 shows the prior art approach.

FIG. 2 shows a primary sterile fluid container (typically a flexible bag, as shown, but perhaps a bottle or other sterile vessel) [1] suspended from a hanger [2] and connected to a primary catheter tube [3] having a primary air lock [4] allowing air to escape the catheter tube before the air enters the patient's blood stream. The hanger [2] also suspends a secondary sterile fluid container [5].

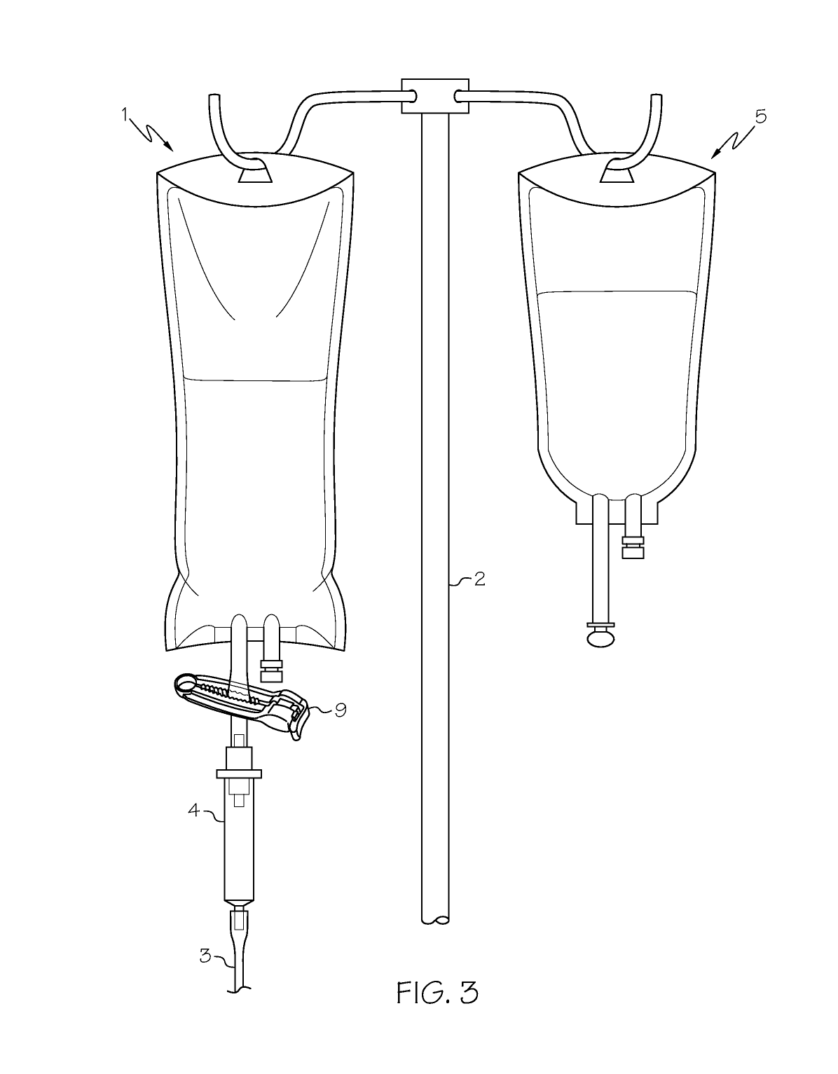

FIG. 3: From the starting configuration exemplified in FIG. 2, one clamps shut the primary catheter tube [3] using a catheter tube clamp [9]. The clamp may be an autoclavable acetal tubing clamp Model No. 1795, commercially available from U.S. Plastic Corp. (Lima, Ohio), as illustrated in FIG. 3. This clamp locks closed and opens when the locking tab is released. This type of clamp can be attached at any point on tubing without disconnecting tubing. When they are not in use, this type of clamp can be clipped to the tubing without restricting flow. The clamp jaws are 27 mm (1 1/16″) long, and the total clamp length is 59 mm (2 5/16″) long. These jagged jaws will close intravenous tubing with a wall of 0.82 mm ( 1/32″) or thinner. Other clamp types are functionally equivalent as long as they close the catheter tube, are removable and are clean.

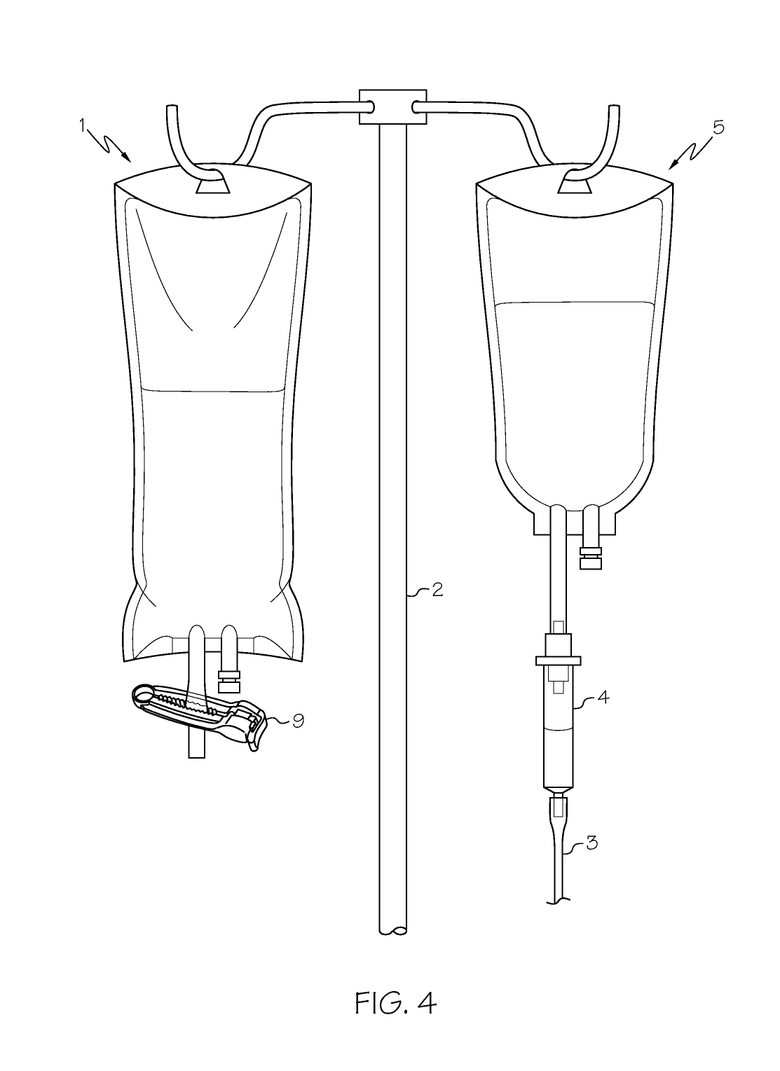

FIG. 4: The opening tube for the secondary sterile fluid container [5] is then opened and the primary catheter tube [3] having a primary air lock [4] is removed from the primary sterile fluid container [1] and connected to the secondary sterile fluid container [5].

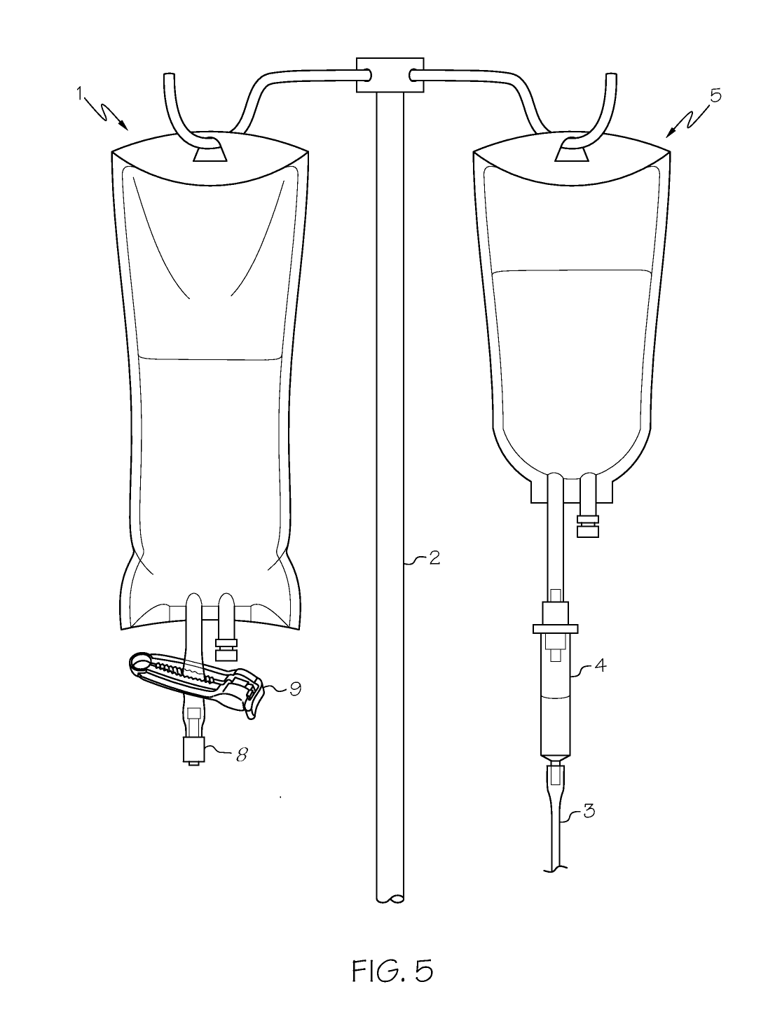

FIG. 5: The opening of the primary sterile fluid container [1] is plugged with a sterile plug [8]. The sterile plug [8] prevents contamination from entering the exposed primary sterile fluid container [1] opening. The opening of the primary sterile fluid container

may preferably be plugged before the secondary sterile fluid container [5] is connected to the primary catheter tube [3] having a primary air lock [4], to minimize the time the primary sterile fluid container [1] opening is exposed to air and to potential contamination.

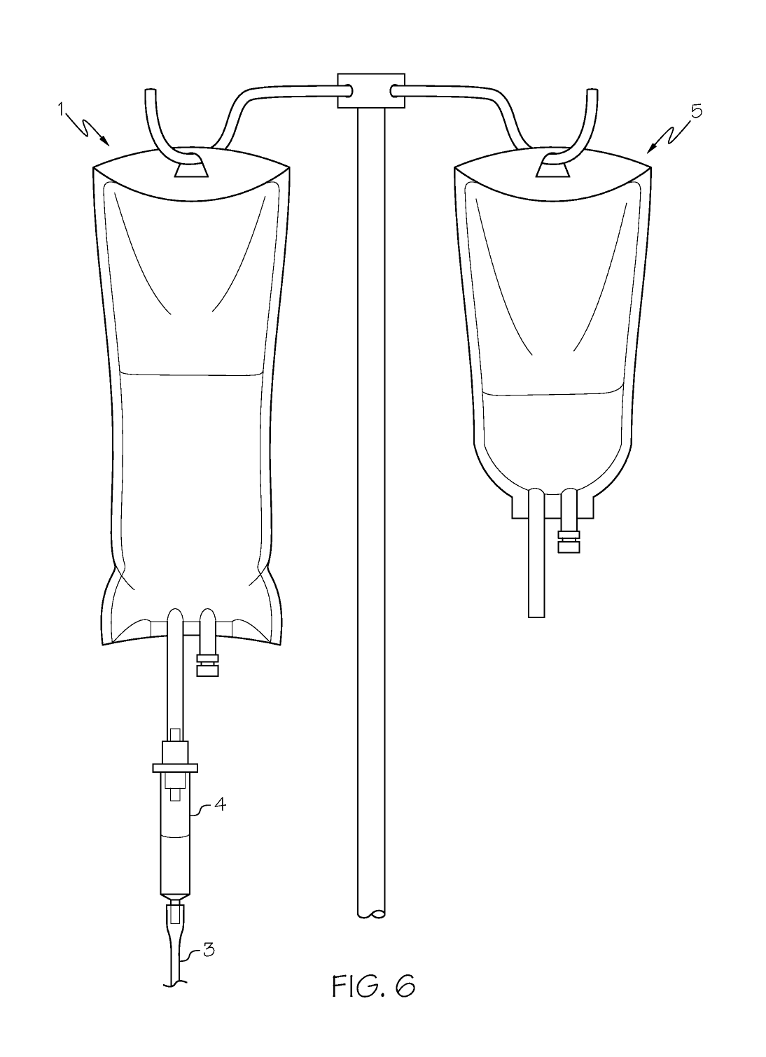

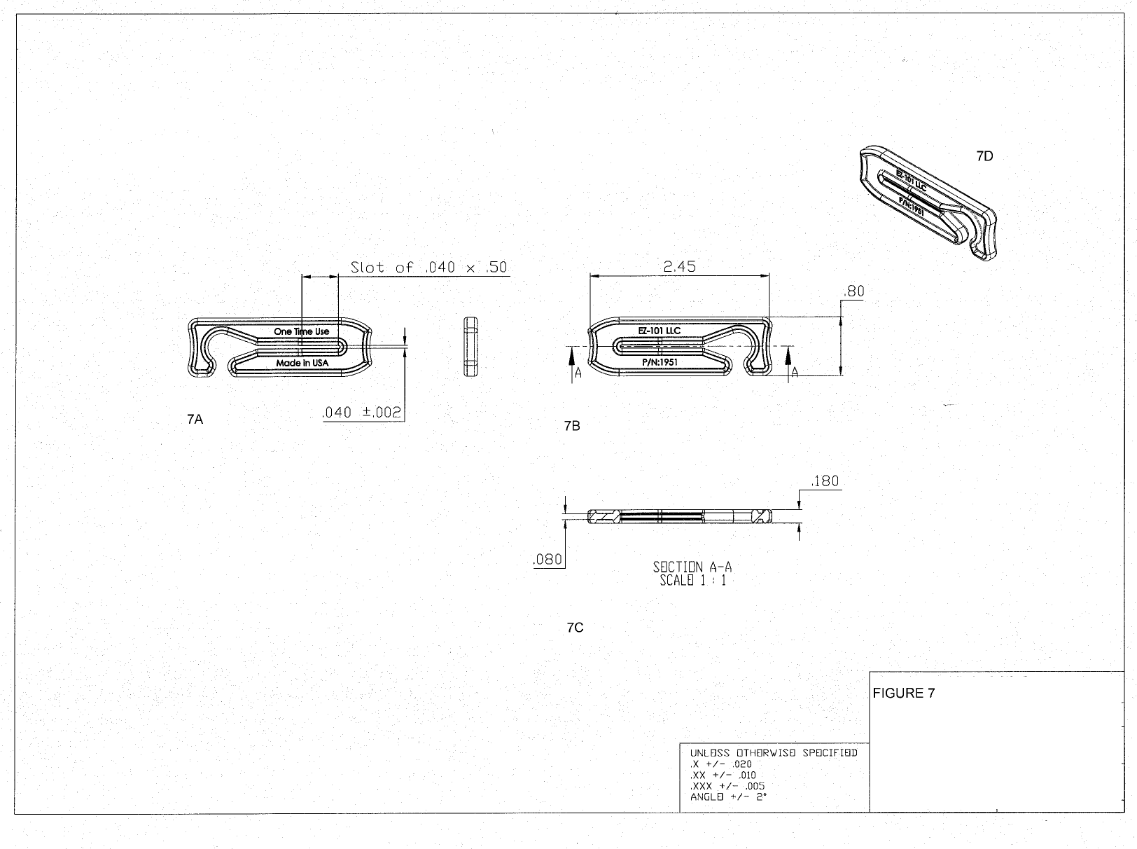

FIG. 6: When the secondary sterile fluid container [5] is empty, the sterile plug [8] is removed from the primary sterile fluid container [1], the primary catheter tube [3] having a primary air lock [4] is disconnected from the secondary sterile fluid container [5] and re-connected to the primary sterile fluid container [1]. Once the primary catheter tube [3] having a primary air lock [4] is re-connected to the primary sterile fluid container [1], the catheter tube clamp [9] is removed.

Given this disclosure, variants of the method described and illustrated above will be readily apparent. For example, the secondary sterile fluid container [5] as exemplified in the Figures is not opened before it is connected to the primary catheter tube [3] having a primary air lock [4]. Alternatively, the secondary sterile fluid container [5] may first be prepared by opening it and protecting the opening with a removable a sterile plug [8]. In this variant, the removable a sterile plug [8] is removed before connecting the primary catheter tube [3] having a primary air lock [4], and the removable a sterile plug [8] may be re-used to seal the primary sterile fluid container [1].

My method may be made easier by providing a unitary kit containing a catheter tube clamp [9] together with a removable a sterile plug [8]. The kit may optionally further contain a primary or secondary sterile fluid container containing e.g., normal saline or a pre-filled intravenous bag pre-filled with a therapeutic liquid.

I intend the legal coverage of my patent to be defined not by the specific examples recited here, but by the legal claims approved by the Patent Office (and permissible legal equivalents of the legal claims). I intend my patent to cover, for example, the process of:
1. Suspending a primary sterile fluid container having an opening from a hanger,
2. Connecting the primary sterile fluid container opening to a primary catheter tube,
3. Suspending a secondary sterile fluid container from the hanger,
4. Clamping shut the primary catheter tube using a catheter tube clamp,
5. Disconnecting the primary catheter tube from the primary sterile fluid container opening,
6. Covering the primary sterile fluid container opening with a sterile plug,
7. Connecting the primary catheter tube to an opening in the secondary sterile fluid container and
8. Removing the catheter tube clamp from the primary catheter tube.
   I also intend my patent to cover a kit including:
1. a catheter tube clamp and
2. a sterile plug, and
3. optionally a sterile fluid container.
   I also intend my patent to cover the method of providing such a kit, and more specifically the method of providing such a kit for the aforementioned method.

Given this general disclosure, the artisan may make variations on this. For example, while the Figures here illustrate a sterile plug [8], commercially-available sterile tape sealant is equivalent to seal the catheter line. Alternatively, albeit more expensively, one could use a catheter tube clamp [9] for this purpose.

As a catheter tube clamp [9], one may use a hinged closable clamp as illustrated. Non-hinged C-clamps are commercially available, and offer the advantage of being able to be easily applied and removed with one hand. I have found, however, that commercially-available catheter tube C-clamps fail to provide a leak-proof seal to the catheter tube. This is apparently because the opening of the C-clamp is slightly too large to adequately compress the tubing, or the depth of the clamp is inadequate and thus allows torsional deformation which reduces the compression pressure on the tube. I thus here provide designs for a C-clamp of my own design, which is properly dimensioned to completely close off catheter tubing.

I claim:

1. A method comprising:
a. Providing a primary sterile fluid container having an opening, and then connecting the primary sterile fluid container opening to a primary catheter tube, and then clamping shut the primary catheter tube using a catheter tube clamp, and then disconnecting the primary catheter tube from the primary sterile fluid container opening and then covering the primary sterile fluid container opening with a sterile cover,
b. Providing a secondary sterile fluid container, and then connecting the primary catheter tube to an opening in the secondary sterile fluid container, and then removing the catheter tube clamp from the primary catheter tube.

2. The process of claim 1 where said sterile cover is a tape.

3. The process of claim 1 where said sterile cover is a plug.

4. The process of claim 1 where said catheter tube clamp is a C-clamp.

5. The process of claim 4 where said C-clamp is as illustrated on FIG. 7.

6. A multi-line catheter management kit comprising:
a. A catheter tube clamp, and
b. A catheter tube sterile cover.

7. The kit of claim 6, where said sterile cover is a tape.

8. The kit of claim 6, where said sterile cover is a plug.

9. The kit of claim 6, where said catheter tube clamp is a C-clamp.

10. The kit of claim 9, where said C-clamp is as illustrated on FIG. 7.

11. The kit of claim 6, further comprising:
c. catheter tubing.

12. A method comprising: providing a multi-line catheter management kit comprising a catheter tube clamp, a catheter tube sterile cover and catheter tubing.
